(19) United States
(12) Patent Application Publication
(10) Pub. No.: US 2026/0137600 A1
(43) Pub. Date: May 21, 2026

(54) FEED AND FLUSH DELIVERY DEVICE

(71) Applicant: VESCO MEDICAL LLC, COLUMBUS, OH (US)

(72) Inventors: Christopher O'Keefe, Columbus, OH (US); Rachel Kunzweiler, Columbus, OH (US); Aaron Szabo, Columbus, OH (US)

(21) Appl. No.: 19/449,029

(22) Filed: Jan. 14, 2026

Related U.S. Application Data
(62) Division of application No. 16/743,152, filed on Jan. 15, 2020.

(51) Int. Cl. A61J 15/00; A61M 5/14; A61M 5/142; A61M 5/168

(52) U.S. Cl. CPC A61J 15/0092; A61M 5/1408; A61M 5/14232; A61M 5/16813; A61M 2005/1403

(57) ABSTRACT

Feed and flush delivery devices are described for use in switching between delivery of enteral feeding fluid and flushing/hydration fluid to a patient, with both fluids being delivered through the same closed system. The delivery devices are simpler, easier to use, and are more cost effective than known prior art valves and control devices. Independent feed and flush lines are typically received by the delivery device, which merges the two lines into a single end delivery line so that either feeding or flushing fluid, but not both at the same time, can be conveyed through the end line, as desired and selected by the user. The delivery device is loaded onto the pumping mechanism of an enteral infusion pump, and can be part of a cassette which is used in combination with a particular brand of infusion pump.

### CROSS REFERENCES TO RELATED APPLICATION

The present application is a Divisional application claiming the benefit of pending U.S. application Ser. No. 16/743,152, filed Jan. 15, 2020, the disclosure of which is incorporated herein by reference in its entirety.

### FIELD OF THE INVENTION

The present invention relates generally to the delivery of enteral nutrition and fluids to a patient, and in particular to delivery devices for engaging an infusion pump and safely delivering flow through a selected enteral feed line or flush line.

### BACKGROUND OF THE INVENTION

Enteral tube feeding uses the gastrointestinal (GI) tract to deliver nutrition and fluids to patients through an enteral feeding tube, and is typically considered in patients who have a functional, accessible gastrointestinal tract but who are malnourished, have an inadequate oral intake, or are at risk of malnutrition. The aim is to optimize nutritional intake and to improve or maintain nutritional status. Feeding via a nasogastric (NG) tube or a naso-jejunal (NJ) tube is typically done for short term needs (less than 4 weeks), and feeding via more invasive routes is considered for long term needs (longer than 4 weeks), which include gastrostomy tubes (PEG, G-tube) and jejunostomy tubes (PEJ, J-tube) inserted percutaneously by a doctor through a small incision in the patient's abdominal wall.

Enteral tube feeding is commonly administered via an enteral feeding set, also referred to as an administration feeding set, which typically includes a bag or reservoir which serves as a source for a particular enteral fluid or nutritional product, and tubing for connecting the bag to an enteral feeding pump such as a peristaltic infusion pump. A section of the feeding set tubing is typically loaded onto the infusion pump by placing the tubing in a stretched condition around rollers of the pump. The feeding set tubing which is downstream of the infusion pump is then connected to the patient's enteral feeding tube. The administration feeding set is then ready for operation, and the infusion pump can be programmed and/or activated to automatically deliver the enteral fluid to the patient at a controlled rate.

Enteral nutritional products are typically in the form of a relatively thick, flowable liquid or fluid, and many types are available for a variety of replacement and supplemental feeding requirements. In many instances it is desirable to sequentially deliver two different types of enteral nutritional products to a patient in order to meet specific dietary or medicinal needs. For example, delivery of a first type of enteral nutritional fluid may be followed by delivery of a second type of fluid containing either different nutritional products or medications. In addition, it is often necessary to provide hydration fluid such as water to the patient. Water is also often necessary to flush nutritional product residue from the tubing, to check for and maintain tube patency, and to prevent clogging of the feeding tube. It is recommended that feeding tubes (G-tube, PEG tube, etc.) be flushed regularly with water (or sterile water if appropriate) prior to and after nutritional feeding or medication delivery, and regularly in between tube use. As a result, “feeding fluids” and “flushing/hydration fluids” are often sequentially administered through the same feeding tube.

Different administration feeding sets containing different nutritional and medical products often look similar to the naked eye. Therefore, it is very important that the specific enteral fluid desired, for example, either feeding fluid or flushing/hydration fluid, is properly and accurately delivered to the patient. While specialized valve mechanisms for safely switching between differing types of enteral fluids are known, they can be complex in design and complicated to use, requiring specialized training for healthcare personnel. It would be advantageous if the nurse or doctor were able to switch between delivering two different types of enteral fluids (e.g. between feed and flush lines) efficiently and safely without the need for complex loading maneuvers and complicated valve control mechanisms.

In light of the above, it would be useful to provide an enteral fluid delivery device for use with known peristaltic infusion pumps which can quickly switch between delivering a first enteral fluid and second enteral fluid within the same closed system. It would also be beneficial if such a delivery device could be easily employed by medical personnel having a variety of skill levels.

### SUMMARY OF THE INVENTION

Accordingly, the present invention provides a plurality of embodiments of an enteral fluid delivery device for switching between delivery of a first enteral fluid (i.e. “feed” fluid) and a second enteral fluid (i.e. “flush” or “hydration” fluid) through a single end delivery line.

A first aspect of the invention provides a feed and flush delivery device for connecting an administration feeding set to a peristaltic infusion pump to allow switching between feeding fluid and flushing fluid within the same closed system, the delivery device comprising: a feed line comprising tubing fluidly connected to an enteral feeding fluid; a flush line comprising tubing fluidly connected to a flushing or hydration fluid; an end delivery line fluidly connected to both the feed line and the flush line; and a divider including two inlet ports and a single outlet port for fluidly connecting the feed line and the flush line to the end delivery line, wherein the infusion pump includes a motor for powering a rotatable shaft, the rotatable shaft having at least one rotor mounted thereto, the at least one rotor including a plurality of rollers circumferentially spaced about the shaft for making contact with either the feed line, the flush line or the end delivery line and creating peristaltic fluid flow through the delivery device, and wherein the delivery device in combination with the infusion pump allows for selective delivery of either the feeding fluid or the flushing fluid through the end delivery line.

A second aspect of the invention provides feed and flush delivery device for connecting an administration feeding set to a peristaltic infusion pump to allow switching between delivery of a first enteral fluid and a second enteral fluid to a patient within the same closed system, the delivery device comprising: a first enteral fluid line fluidly connected to a first enteral fluid; a second enteral fluid line fluidly connected to a second enteral fluid; an end delivery line fluidly connected to both the first enteral fluid line and the second enteral fluid line; a divider having two inlet ports and a single outlet port for fluidly connecting the first enteral fluid line and the second enteral fluid line to the end delivery line; and a window for providing contact points between the constricting instruments and the enteral fluid lines, wherein the infusion pump includes a motor for powering a rotatable shaft, the rotatable shaft having single uni-directional rotor including a plurality of rollers circumferentially spaced about the shaft for making contact with the end delivery line and creating peristaltic fluid flow through the delivery device, and wherein the delivery device in combination with the infusion pump allows for selective delivery of either the first enteral fluid or the second enteral fluid through the end delivery line.

The delivery device can be in the form of a cassette which can be used in combination with a particular variety of peristaltic infusion pump, and the infusion pump can be programmed to deliver continuous or intermittent feeding, as well as provide automatic flushing/hydration capability. The nature and advantages of the present invention will be more fully appreciated after reviewing the accompanying drawings, detailed description, and claims.

### BRIEF DESCRIPTION OF THE DRAWINGS

The accompanying drawings illustrate embodiments of the invention and, together with a general description of the invention given above, and the detailed description given below, serve to explain the principles of the invention.

FIG. 1 is a perspective view of a peristaltic infusion pump and a schematic representation of a delivery device according to the general principles of the present invention;

FIG. 2 is a perspective view of one embodiment of a delivery device according to the present invention which uses two rotors to separately control the flow of a feed line and a flush line;

FIG. 3 is a perspective view of one embodiment of a delivery device according to the present invention which uses a single rotor and a pair of check valves to separately control the flow of the feed line and the flush line;

FIG. 4 is a perspective view of another embodiment of a delivery device according to the present invention;

FIGS. 5A and 5B are perspective views of another embodiment of a delivery device according to the present invention;

FIGS. 6A and 6B are perspective views of another embodiment of a delivery device according to the present invention;

FIGS. 7A and 7B are perspective views of a cassette embodiment of the delivery device according to the present invention.

### DETAILED DESCRIPTION OF THE INVENTION

Feed and flush delivery devices as described herein can be used to alternatively deliver enteral feeding fluid or flushing/hydration fluid to a patient, with both the feeding fluid and the flushing fluid being delivered through the same closed system. Independent feed and flush lines are typically received by the inventive delivery device, which then merges the two lines into a single end delivery line so that either feeding or flushing fluid, but not both at the same time, can be conveyed through the end line, as desired and selected by the user.

As used herein, the terms “connector(s)”, “connection(s)” and “connector device(s)” mean a device for reversibly connecting a peristaltic infusion pump to a feeding set. A connector is typically one element of a cassette, as defined herein, and can provide a simple “infusion pump-to-feeding set” integration or “keying” system for connecting a single use feeding set to a matching reusable infusion pump, ensuring proper operation of both.

The term “cassette” as used herein means a portion of a feeding set, specifically, a connector in combination with small pump tubing segments, as defined herein. In use, the connector portion of a cassette can be locked into a receiving portion of a peristaltic infusion pump housing, which aligns the pump tubing segment for engagement with the pumping mechanism.

The phrase “pump tubing segment(s)” as used herein refer to a piece or segment of tubing that is fluidly continuous with a feed and/or flush line and which is coupled with a connector to form a cassette. Pump tubing segments can be used fluidly connect to feed and flush lines as well as to end delivery lines within a feeding set, and depending on the embodiment disclosed herein can also be used as part of a cassette to be engaged by the rollers of a peristaltic infusion pump.

As known in the art, a “peristaltic infusion pump” or “infusion pump” typically includes a mechanical pump with an array of spaced apart rollers mounted to a motor-driven rotor for sequentially making contact with and occluding either feed tubing, flush tubing, or end delivery line tubing, or with a pump tubing segment element of a cassette which is fluidly connected to either feed tubing, flush tubing, or end delivery line tubing.

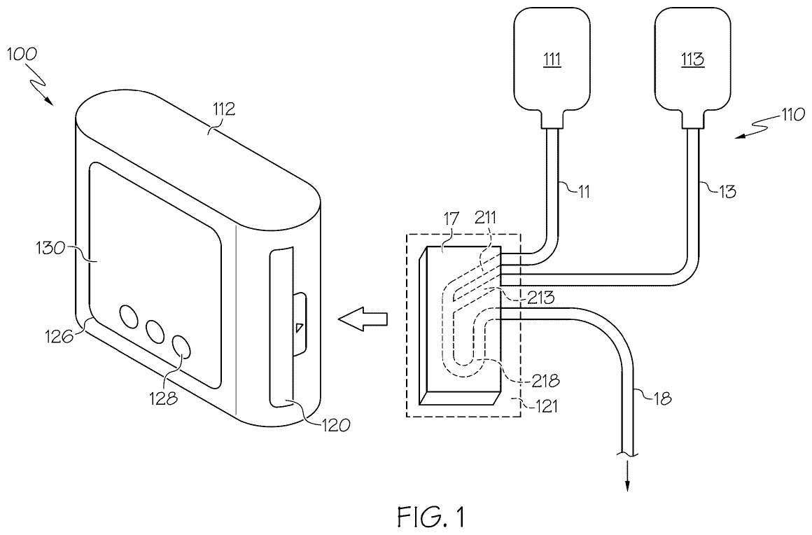

The inventive delivery device is intended for connection to an enteral feeding pump such as a peristaltic infusion pump. A typical prior art enteral feeding pump that can be used with the present invention is shown in FIG. 1, specifically, a peristaltic infusion pump 100 is illustrated having a housing 112 constructed to allow mounting of an administration feeding set 110. The feeding set 110 includes a first enteral nutritional fluid source 111 (e.g. “feeding fluid”) and a second enteral nutritional fluid source 113 (e.g. “flushing/hydration fluid”), as well as two source lines or tubes 11, 13. Loading of administration feeding set tubing onto a pump rotor is well known in the art, and is typically performed by engaging or otherwise stretching or tensioning a portion of the tubing against, around or about one or more of the pump rollers. In this manner, a set amount of fluid can be caused to flow to the patient at a desired volumetric rate. For example, peristaltic infusion pumps are typically set to deliver enteral fluid at rates between 5 ml and 600 ml per hour. Feeding fluids are usually started at a low rate (about 25-50 ml/hr) and then later increased in stages to about 100 to 150 ml/hour after tolerance is demonstrated.

The inventive delivery device is intended to allow flow of only one of the two fluid sources 111, 113. More specifically, and as shown in the figures and described in more detail below, the inventive delivery device can include a section of a first enteral fluid line 11 (e.g. the “feed line”) which will pull from a first enteral fluid source 111 (e.g. “feeding fluid”), and a section of a second enteral fluid line 13 (e.g. the “flush line”) which will pull from a second enteral fluid source 113 (e.g. “flushing/hydration fluid”). The feed and flush lines 11, 13 are connected to a divider 17 which merges them into a single end delivery line 18 which then continues downstream to the patient. Various embodiments of the inventive delivery device are disclosed herein, each providing an alternative to known methods and devices for switching between a feed line and a flush line.

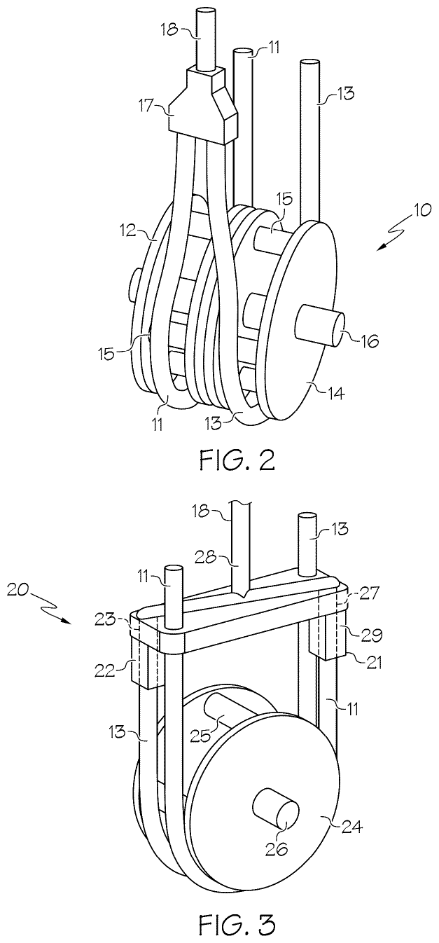

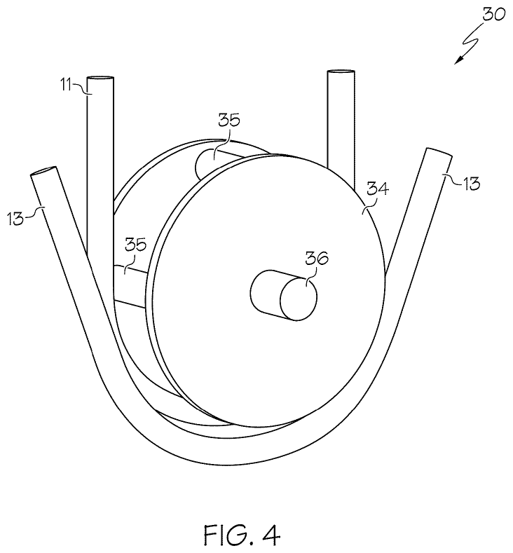

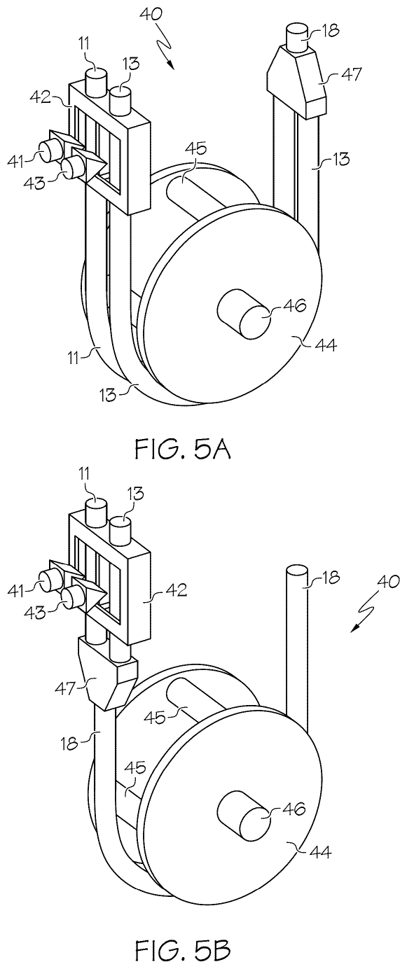

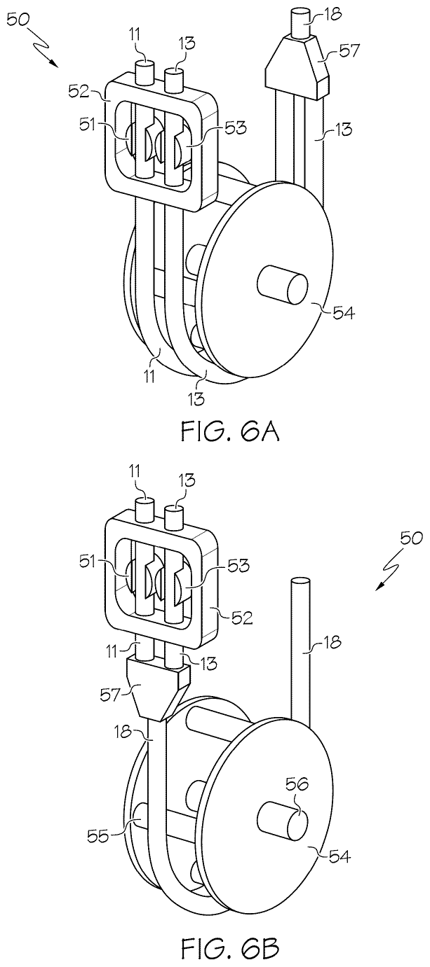

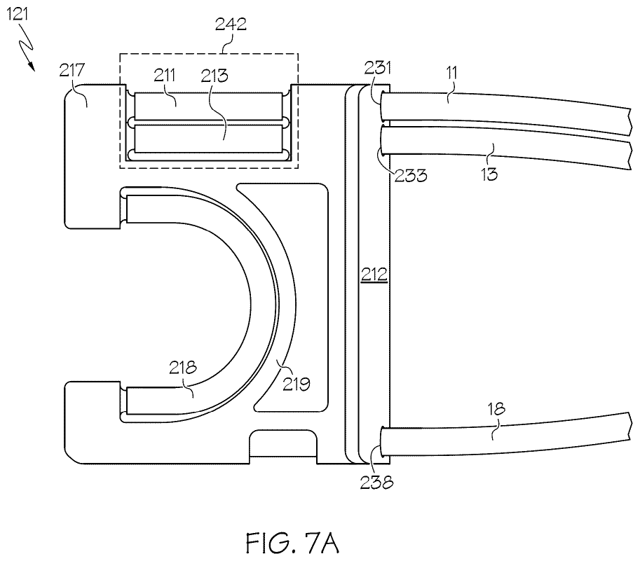

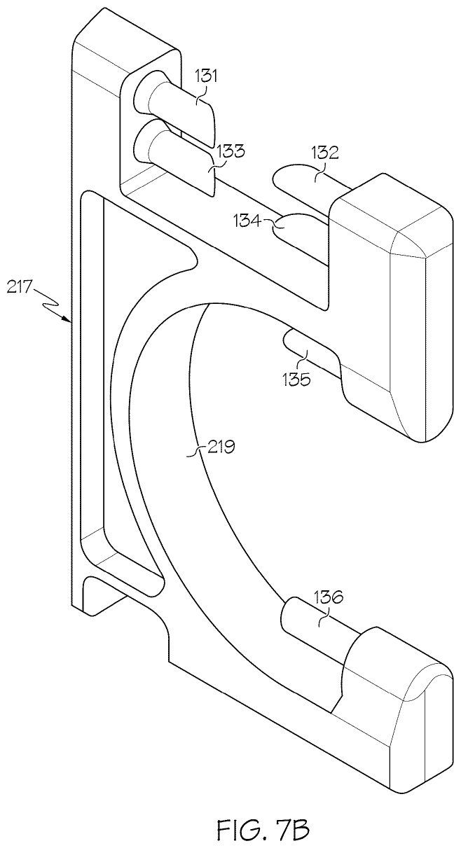

A schematic representation of the inventive delivery device 121 is shown in FIG. 1 generally including segments or portions of the feed and flush lines 11, 13, a divider 17, and a single end delivery line or tube 18, all of which are adapted for fluid flow therethrough. A variety of embodiments of the inventive delivery device are illustrated in the figures, i.e. as a cassette 121 in FIGS. 7A/B, and as device 10 in FIG. 2, device 20 in FIG. 3, device 30 in FIG. 4, device 40 in FIGS. 5A/B, and device 50 in FIGS. 6A/B. The flow divider (see divider 17 in FIG. 2, divider 27 in FIG. 3, divider 47 in FIGS. 5A/B, divider 57 in FIGS. 6A/B, and connector 217 in FIGS. 7A/B) includes two inlet ports and a single outlet port for fluidly connecting the feed line 11 and the flush line 13 to the end delivery line 18. The feed line 11 is in fluid communication with or otherwise fluidly connected to the first/feeding fluid source 111 and the divider 17, and the flush line 13 is in fluid communication with the second/flushing fluid source 113 and the divider. The divider 17 is in fluid communication with the feed and flush lines as well as the end delivery line 18.

A receiving port 120 in the housing 112 of the infusion pump 100 can provide access to a peristaltic infusion pump, as is known in the art. The infusion pump can include at least one motor-driven rotor mounted on a rotatable shaft and having a plurality of rollers. A single rotor is illustrated in FIGS. 3, 4, 5A/B and 6A/B; however, as seen in FIG. 2, the shaft 16 can also include dual rotors 14, 15. Each rotor typically has a plurality of rollers mounted thereon (illustrated as element 15 in FIG. 2, element 25 in FIG. 3, element 35 in FIG. 4, element 45 in FIGS. 5A/B and as element 55 in FIGS. 6A/B) which engage the tubing of the delivery device so that, when in motion, a set amount of fluid flows to the patient at a desired volumetric rate. For example, contact with the rollers is made via the feed and flush lines 11, 13 in FIGS. 5A and 6A, via the end delivery line 18 in FIGS. 5B and 6B, and via pump tubing segment 218 in FIG. 7A.

Looking again at FIG. 1, the pump housing 112 typically has a user interface 126 with a display screen 130 on the front for displaying information about the status and operation of the pumping mechanism inside. The housing typically further includes buttons 128 and/or lights for use with the display screen 130 to facilitate exchanging information between the feeding pump and the user. As an example, the display screen 130 may be an LCD glass panel with a graphical user interface having a touch screen by which the user can provide input information. The infusion pump 100 can be connected to an external source of electrical power, or alternatively/additionally a battery may be disposed in the housing for providing electrical power to the pump motor, as is known in the art.

According to the various embodiments of the present invention, a user can connect or otherwise mount the inventive delivery device to the rollers of an infusion pump 100 via the feed and flush lines 11, 13 or via the end delivery line 18 for delivery of the prescribed nutritional fluid stored inside containers 111 and/or 113 to the patient. The delivery device can be self-contained, e.g. in the form of a cassette (121, see FIGS. 1 and 7A/B), which can be manufactured to include a predetermined length or segment 211, 213, 218 of the feed and flush lines and end delivery line collectively referred to herein as “pump tubing segments”. As illustrated in FIG. 1, the cassette 121 can be reversibly inserted into the infusion pump housing via the receiving port 120 for engagement with the pump located inside the housing 112. The tubing of the delivery device can be aligned so that portions of the feed and flush lines 11, 13 and/or a portion of the end delivery line 18 engages the rollers of the pump rotor. For example, as illustrated in FIG. 2, the feed and flush lines 11, 13 can engage dual rotors 12, 14; alternatively, as illustrated in FIGS. 3, 5A and 6A, both the feed and flush lines 11, 13 can engage a single rotor 24; see also FIGS. 5B and 6B in which the end delivery line 18 engages the rotor, and FIG. 7A in which a smaller pump tubing segment 218 can engage the pump rotor as part of a cassette 121.

The delivery device 10 shown in FIG. 2 is referred to as a “dual rotor” design because it uses two rotors, namely, a first pump rotor 12 and a second pump rotor 14 to separately control the flow of the feed line 11 and the flush line 13. As shown in FIG. 2, the dual rotors 12, 14 share a common shaft 16. Each rotor 12, 14 has a plurality of bearing sleeves or rollers 15 circumferentially spaced about the shaft 16 to compress a segment of tubing, i.e. either the feed line 11 or the flush line 13, which has been contacted by it. As shown, a section of tubing for both the feed line 11 and the flush line 13 is contacted by, i.e. wrapped, stretched or otherwise tensioned around its respective rotor 12, 14. The tension created by contacting the tubing with the rollers 15 is great enough to fully collapse the feed or flush tubing 11, 13, such that no flow is permitted in the respective line 11, 13 when its rotor 12, 14 is not turning, but peristaltic fluid flow is created through the tubing 11, 13, 18 when the rotor is turning.

As illustrated in FIG. 2, both the first (e.g. feed) line 11 and the second (e.g. flush) line 13 are combined into a single delivery line 18 via connection to a Y-type flow divider 17, the divider having two inlet ports and a single outlet port, such that fluid within either line 11 or line 13 can be selectively delivered through the single end delivery line 18 to the patient. When one rotor turns (for example, first pump rotor 12) there will be flow through its respective tubing line 11 by means of a peristaltic pumping motion created by the actuated rotor. The second pump rotor 14 remains stationary and so collapses and does not permit fluid flow through contacting line 13. As the first pump rotor 12 is selectively turned by the power source, its rollers 15 act on the tubing 11 to advance evenly spaced occlusions there along, so that a specific volume of the first enteral fluid is positively displaced between successive occlusions through the tubing by the pumping mechanism; as such, the fluid within line 11 (or line 13, if the second pump rotor 14 is being activated) is then delivered through the single end delivery line 18 to the patient, thereby delivering a predetermined amount of either the first enteral fluid or the second enteral fluid, as desired, over a period of time. Each rotor 12, 14 can be powered by its own power source within the peristaltic infusion pump to separately control flow through the two lines 11, 13; alternatively, the dual rotors can be powered by a single power source having a clutch mechanism as is known in the art, which transfers power to the desired rotor.

FIG. 3 illustrates another embodiment of the inventive delivery device according to the present invention, referred to herein as a “check valve” design 20. This embodiment employs a single, bi-directional rotor 24 mounted on shaft 26, and additionally utilizes a first one-way check valve 21 for the feed line 11, and a second one-way check valve 22 for the flush line 13. One-way check valves are well known in the art and permit flow in only a single direction. They can include an in-line check valve, a duckbill valve, an umbrella valve, a ball-check valve, a diaphragm check valve, a swing check valve, a stop-check valve, a lift-check valve, etc.

Each check valve 21, 22 shown in FIG. 3 is normally in the closed position, and opens when the fluid pressure within either fluid line 11, 13 increases to allow flow through it. The check valves 21, 22 also have the capability to resist flow in the opposite direction, so that backflow from the divider 27 into the enteral lines 11, 13 is not permitted. Specifically, fluid passing through either check valve 21, 22 can only pass through one of two inlet ports of the rectangular divider 27, namely, a first inlet port 23 and a second inlet port 29, and on through a single outlet port 28, which is connected to the end delivery line 18. Reverse flow through the check valves 21, 22 is not permitted.

Looking at FIG. 3 it can be appreciated that rotation of the bi-directional rotor 24 in a first (e.g. counter-clockwise) direction will increase fluid pressure in line 11 to open check valve 21 and allow the first enteral fluid (i.e. feeding fluid) to pass through the divider 27 then out through the end delivery line 18 and on to the patient. During such counter-clockwise rotation the check valve 22, which is connected to inlet port 23, prevents back flow of feeding fluid from the divider 27 into the flush line 13. Conversely, during rotation of the bi-directional rotor 24 in a second (e.g. clockwise) direction, check valve 22, which is connected to flush line 13, will be in the open position and allow the second enteral fluid (i.e. flushing/hydration fluid) to pass through the divider 27 then out through outlet port 28 and end line 18 and on to the patient, while the check valve 21 connected to inlet port 29 prevents back flow of flushing fluid into the feed line 11. The function of the check valves 21, 22 (open or closed) is therefore determined by the direction of the rotor 24, which also determines which fluid (i.e. feed or flush) is delivered via the end delivery line 18.

FIG. 4 illustrates another embodiment of the inventive delivery device, referred to herein as a “slack line” design 30. This delivery device 30 utilizes a single unidirectional rotor 34 to separately control the feed and flush lines, 11, 13, and allows for the rotor 34 to turn and perform pumping action while tensioning a first enteral fluid line (e.g. “feed line”) 11 while a second enteral fluid line (e.g. “flush line”) 13 is given slack so that the fluid flow in the slack line remains stagnant. This “slack line” design includes a unidirectional rotor 34 with a plurality of (as shown, a set of four) rollers 35 circumferentially spaced about a shaft 36, as described above. Pumping action for one of either the feed line 11 or the flush line 13 is bypassed when that line is given enough slack to lose contact with the rollers 35. In use, for example, if the desired user action is to pump enteral feeding fluid through the feed line 11, then the infusion pump is programmed to put tension on the feed line 11 while the flush line 13 is given slack. Alternatively, if the desired user action is to pump flushing fluid through the flush line 13 then the system is programmed to put tension on the flush line 13 while the feed line 11 is given slack. Downstream of the rotor 34 both lines 11, 13 eventually combine into a Y-type flow divider (not shown, but see divider 17 described above) and flow through the single end line (18) and on to the patient. When a particular line 11, 13 is placed in the slack position, gravitational backflow can be prevented by way of including a low pressure valve in line with the tubing, for example, a duckbill valve. Duckbill valves are one-way valves commonly used in medical applications as backflow prevention devices, and generally comprise elastomeric lips in the shape of a duckbill to prevent backflow and allow only forward flow.

FIGS. 5A and 5B illustrate embodiments of a delivery device 40 referred to herein as a “linear pinch” design. Like the slack line design described above, each linear pinch embodiment utilizes a single unidirectional rotor 44 having a set of rollers 45 circumferentially spaced about a shaft 46. Either tubing 11, 13 (FIG. 5A) or tubing 18 (FIG. 5B) can be contacted by the rollers 45. A rectangular element or window 42 presents, houses or otherwise borders sections or portions of the first (feed) and second (flush) tubing lines 11, 13, to provide stable contact points for a set of wedges or pinching instruments 41, 43. When actuated, the pinching instruments 41, 43 prevent flow through their respective tubing lines 11, 13. The window 42 can be part of the infusion pump housing, or part of a cassette that is inserted into the pump housing, and the pinching instruments can be controlled by the infusion pump. A cassette 121, as described above for FIG. 1 and below for FIGS. 7A and 7B, can be manufactured to include a window 242 which holds a “pump tubing segment”, i.e. as shown in FIG. 7A, a predetermined length of the feed line 211, a predetermined length of the flush line 213, and a predetermined length of the end delivery line 218. The cassette can function to permit identification and loading of the tubing by the infusion pump upon loading of the cassette within the receiving port 120 of the pump, rendering the administration feeding set ready for operation upon proper insertion of the cassette into the infusion pump.

In both FIG. 5A and FIG. 5B the feed pinching instrument 41 and the flush pinching instrument 43 are shown centered within the window 42. In FIG. 5A a first (e.g. feed) line 11 and second (e.g. flush) line 13 are both contacted by the rollers 45 of the rotor 44 and then afterwards are combined into a single delivery line 18 via divider 47. In contrast, in FIG. 5B the feed line 11 and the flush line 13 are combined into the single delivery line 18 via divider 47 prior to reaching the rotor 44, and the single end delivery line 18 is then contacted by the rollers 45. In either embodiment, selective actuation during use of the infusion pump of only one of the two pinching instruments 41, 43 selectively allows fluid flow through a desired feed or flush line.

More specifically, rotation of the unidirectional rotor 44 in combination with deflection or pinching of one of the tubing lines 11, 13 can prevent flow of fluid through the pinched line. For example, both feed and flush tubing lines 11, 13 can normally remain open, but the tension created by the rollers 45 is great enough to fully collapse the feed or flush tubing such that no flow is permitted when the rotor is not turning. Thus during a feed cycle the feed line 11 can remain open and the flush line 13 can be pinched by its respective pinching mechanism 43. This is done by the pinching mechanism moving in a linear manner such that the pinching mechanism applies force in the perpendicular direction to the linear direction of the flush tubing 13. Conversely, during a flush cycle the flush line 13 can remain in the open position and the feed line 11 can be pinched by its respective pinching mechanism 41. The force exerted by the pinching mechanisms 41, 43 can be applied by manual control, pneumatic control and/or electronic control.

In addition to pinching a tubing line to prevent flow, the deflection of the tubing 11, 13 by the pinching instruments 41, 43 may alternatively permit fluid to flow through the pinched tubing. In this case, it can be appreciated that the tubing would also include an internal valve or nozzle (not shown) that normally obstructs fluid flow through the tubing. For example, it is well known to include a nozzle within the tubing which is fitted such that a tight seal is created by the nozzle to normally prevent fluid flow, such that pinching of the tubing on or near the nozzle by one of the pinching instruments 41, 43 can alter either the tubing's inner channel or the nozzle itself, so that fluid may now flow through the nozzle and thus also through the tubing. Thus, looking at FIGS. 5A and 5B, upon actuation of the pinching instruments 41, 43 to selectively deform either tubing 11 or 13, the normal obstruction to flow in the pinched tubing is relieved. Fluid flow is then permitted through the pinched line.

FIGS. 6A and 6B illustrate an embodiment of a delivery device 50 referred to herein as a “rotational constriction” design. Like the “slack line” and “linear pinch” designs described above, each rotational constriction embodiment utilizes a single unidirectional rotor 54 having a set of rollers 55 circumferentially spaced about a shaft 56. Tubing 11, 13 (FIG. 6A) or tubing 18 (FIG. 6B) can be contacted by the rollers 55. A rectangular element or window 52 houses or otherwise borders portions of the feed and flush tubing lines 11, 13 to provide stable contact points for a set of rotating instruments, notably a feed rotating instrument 51 and a flush rotating instrument 53. The window 52 can be part of the infusion pump housing, or part of a cassette that is inserted into the pump housing.

In both FIG. 6A and FIG. 6B the rotating instruments 51, 53 are centered within the window 52 and can act to rotationally constrict and collapse either the feed tubing 11 or flush tubing 13 and prevent fluid from flowing through that respective tubing. In FIG. 6A a first (e.g. feed) line 11 and second (e.g. flush) line 13 are both contacted by the rollers 55 of the rotor 54 and then afterwards are combined into a single delivery line 18 via a divider 57 having two inlet ports and a single outlet port. In contrast, in FIG. 6B the feed line 11 and the flush line 13 are combined into the single delivery line 18 via divider 57 prior to reaching the rotor 44, and the single end delivery line 18 is then contacted by the rollers 55. In either embodiment, selective actuation of only one of the two rotating instruments 51, 53 selectively allows fluid flow through a desired feed or flush line.

Looking at FIG. 6A, the first (e.g. feed) line 11 and second (e.g. flush) line 13 are combined into a single delivery line 18 via divider 57 after both lines 11, 13 have been contacted by the rotor 54. In contrast, FIG. 6B illustrates the feed line 11 and the flush line 13 initially combined into the single delivery line 18 prior to reaching the rotor 54, with the single end delivery line 18 then proceeding from the divider 57 to be contacted by the rollers 55. In either embodiment, the rotating instruments 51, 53 are employed to selectively allow fluid flow from one line (i.e. either line 11 or line 13) to the single end delivery line 18, while fluid flow in the other line is temporarily prevented from proceeding past the active rotating instrument.

The cassette 121 shown schematically in FIG. 1 and described above is illustrated in more detail in FIGS. 7A and 7B. FIG. 7A illustrates an embodiment of a cassette 121 which can correspond with the embodiments of the invention illustrated in FIG. 5B and/or FIG. 6B. Specifically, the cassette 121 includes a connector portion 217 and small pump tubing segments 211, 213, 218. Each pump tubing segment 211, 213, 218 corresponds with and is fluidly connected via the connector 217 to its corresponding feed line 11, flush line 13 and end delivery line 18, respectively. The feed line 11 and the flush line 13 enter the cassette via passages 231, 233 which traverse the outer housing 212 side of the cassette 121. Specifically, feed line 11 is fluidly connected to a feed line segment 211 via passage 231, and the flush line 13 is fluidly connected flush line segment 213 via passage 233. Fluid flow through feed and flush line segments 211 and 213 is then directed within the connector portion 217 of the cassette to an end delivery line segment 218, which is then directed within the connector portion 217 to passage 238 and on through the single end delivery line 18.

In use, selective actuation of only one of two pinching instruments (41, 43, described above and illustrated in FIG. 5B) or rotating instruments (51, 53, described above and illustrated in FIG. 6B), which can make contact with pump tubing segments 211 and 213 within a window 242 portion of the connector portion 217, can selectively allow fluid flow through a selected feed line 11 or flush line 13 and restrict flow through the non-selected line. Pump tubing segment 218 is seated within an inner curve 219 of the connector 217, the shape of which can substantially match the physical curvature of the pump rotor (not shown), such that insertion of the cassette 121 into receiving port 120 in the housing 112 of the infusion pump 100 (See FIG. 1) tensions segment 218 around the rotor.

As best seen in FIG. 7B, tubing supports or attachment structures 131, 132, 133, 134, 135, 136, can be included to provide facile and secure attachment of pump tubing segments 211, 213, 218. For example, attachment structures 135 and 136 located inside the opposing ends of the inner curve 219 can provide means to easily and securely connect the free ends of segment 218 to the connector 217. The attachment structures 131-136 are typically hollow, having substantially the same size and diameter as the tubing they connect to. Further, attachment structure 131 is fluidly connected to passage 231 by a pathway through the body of the connector 217, as is attachment structure 133 to passage 233. Similarly, attachment structures 132 and 134 are fluidly connected to attachment structure 135, with the connector 217 thus acting as a flow divider joining the two hollow structures into a single hollow structure, and attachment structure 136 is fluidly connected within the connector 217 to passage 238. Thus, the hollow passages of the attachment structures 131-136 ensure that the inflow tubing 11, 13 and outflow tubing 18 is fluidly connectable to one another via the pump tubing segments 211, 213, 218 of the cassette.

Looking again at FIG. 1, the receiving port 120 of the pump 100 can be designed to match the physical shape of the cassette 121 so that only a particular cassette type can be inserted and locked into the pump 100. This can ensure that only a particular brand of delivery device can be connected to a particular brand of infusion pump. In addition, as is known in the art, an optical reader can be incorporated into the infusion pump to read an imprinted item on the cassette and/or on the tubing segments seated within the cassette. The optical reader can thus verify that a specific infusion pump has been properly loaded with a matching cassette or delivery device, which in turn has been matched with the appropriate administration feeding set, so that the prescribed nutritional fluid is properly delivered. For example, the optical reader can be in the form of an optoelectronic sensor, essentially, a tiny low-resolution video camera.

The various embodiments of the invention as illustrated and described above are simpler, easier to use, and are more cost effective than known prior art valves and control devices. The administration feeding set tubing and the pump tubing segments described herein are typically made of medical grade, deformable polyurethane, PVC, or silicone tubing and provides a fluidic pathway between the enteral fluid sources and the patient.

While particular embodiments of the present invention have been illustrated and described herein in considerable detail, the details regarding these embodiments are not intended to restrict or limit the scope of the appended claims. Accordingly, while only a few such embodiments are particularly described and illustrated herein, it should be understood that the practice of additional modifications and variations of these embodiments, and the equivalents thereof, are within the scope of the invention as recited in the following claims.

Because the instant application is a divisional application, to the extent any amendments, characterizations, or other assertions previously made (in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be revisited.

What is claimed is:

1. A feed and flush delivery device for connecting an administration feeding set to a peristaltic infusion pump to allow switching between feeding fluid and flushing fluid within the same closed system, the delivery device comprising:
a) a feed line comprising tubing fluidly connected to an enteral feeding fluid;
b) a flush line comprising tubing fluidly connected to a flushing or hydration fluid;
c) an end delivery line fluidly connected to both the feed line and the flush line; and
d) a divider including two inlet ports and a single outlet port for fluidly connecting the feed line and the flush line to the end delivery line,
wherein the infusion pump includes a motor for powering a rotatable shaft, the rotatable shaft having at least one rotor mounted thereto, the at least one rotor including a plurality of rollers circumferentially spaced about the shaft for making contact with either the feed line, the flush line or the end delivery line and creating peristaltic fluid flow through the delivery device, and wherein the delivery device in combination with the infusion pump allows for selective delivery of either the feeding fluid or the flushing fluid through the end delivery line.

2. The delivery device of claim 1, wherein the at least one rotor of the infusion pump comprises a single unidirectional rotor, wherein the end delivery line is contacted by the rollers of the unidirectional rotor, the delivery device further comprising:
e) a first pinching instrument for contacting the feed line; and
f) a second pinching instrument for contacting the flush line, wherein actuation of either the first pinching instrument or the second pinching instrument during rotation of the unidirectional rotor prevents fluid flow through one enteral fluid line and allows enteral fluid flow through the other enteral fluid line.

3. The delivery device of claim 2, further comprising a window for providing contact points between the pinching instruments and their enteral fluid lines.

4. A feed and flush delivery device for connecting an administration feeding set to a peristaltic infusion pump to allow switching between delivery of a first enteral fluid and a second enteral fluid to a patient within the same closed system, the delivery device comprising:
a) a first enteral fluid line fluidly connected to a first enteral fluid;
b) a second enteral fluid line fluidly connected to a second enteral fluid;
c) an end delivery line fluidly connected to both the first enteral fluid line and the second enteral fluid line;
d) a divider having two inlet ports and a single outlet port for fluidly connecting the first enteral fluid line and the second enteral fluid line to the end delivery line; and
e) a window for providing contact points between the constricting instruments and the enteral fluid lines, wherein the infusion pump includes a motor for powering a rotatable shaft, the rotatable shaft having single uni-directional rotor including a plurality of rollers circumferentially spaced about the shaft for making contact with the end delivery line and creating peristaltic fluid flow through the delivery device, and wherein the delivery device in combination with the infusion pump allows for selective delivery of either the first enteral fluid or the second enteral fluid through the end delivery line.

5. The delivery device of claim 4, the delivery device further comprising:
g) a first pinching instrument for contacting the first enteral fluid line; and
h) a second pinching instrument for contacting the second enteral fluid line, wherein actuation of either the first pinching instrument or the second pinching instrument during rotation of the unidirectional rotor prevents fluid flow through the first enteral fluid line and allows enteral fluid flow through the second enteral fluid line.

6. The delivery device of claim 5, further comprising a window for providing contact points between the pinching instruments and their enteral fluid lines.
